(19) United States
(12) Patent Application Publication
(10) Pub. No.: US 2010/0318061 A1
(43) Pub. Date: Dec. 16, 2010

(54) Catheter

(75) Inventors: Hugo G. Derrick, Stroud (GB); Mathew D F Stratton, Stroud (GB); Eleanor van der Heijden, Wotton-under-Edge (GB)

Correspondence Address: OLIFF & BERRIDGE, PLC, P.O. BOX 320850, ALEXANDRIA, VA, 22320-4850, US

(73) Assignee: RENISHAW (IRELAND) LIMITED, Swords (IE)

(21) Appl. No.: 12/659,458

(22) Filed: Mar. 9, 2010

Related U.S. Application Data
(63) Continuation-in-part of application No. PCT/GB2009/000737, filed on Mar. 19, 2009.
Application No. PCT/GB2009/000737 is a continuation-in-part of application No. PCT/GB2008/003392, filed on Oct. 8, 2008.

(30) Foreign Application Priority Data
Oct. 8, 2007 (GB) 0719607.4
Feb. 13, 2008 (GB) 0802634.6

(51) Int. Cl. A61L 29/02; A61B 1/07; A61B 1/06; A61M 25/00; A61B 10/02; A61B 18/18; A61F 2/28; A61L 33/02

(52) U.S. Cl. 604/508; 600/182; 600/178; 604/523; 600/567; 606/41; 623/23.61; 427/2.1

(57) ABSTRACT

The invention relates to rigid surgical devices formed from rigid ceramics such as zirconium dioxide. In particular, the invention relates to a neurosurgical catheter formed from extruded zirconium dioxide. The invention also relates to an advancement means for advancing or retracting such a device along an axis of insertion into a patient.

### FIELD OF THE INVENTION

The present invention relates to medical catheters and in particular to neurosurgical catheters for insertion directly into the brain parenchyma of a subject.

### BACKGROUND

There are many situations where there is a requirement to deliver therapeutic agents directly to specific targets within the brain parenchyma using implanted catheters. Furthermore, many of these therapeutic agents will cause unwanted side effects if delivered to healthy parts of the brain. Examples of treating abnormalities of brain function include the acute infusion of Gamma-amino-buturic-acid agonists into an epileptic focus or pathway to block transmission, and the chronic delivery of opiates or other analgesics to the peri-aqueductal grey matter or to thalamic targets for the treatment of intractable pain. Also, cytotoxic agents can be delivered directly into a brain tumour. Intraparenchymal infusion can also be used to deliver therapeutic agents to brain targets that can not be delivered systemically because they will not cross the blood-brain barrier. For example, the treatment of patients with Parkinson's disease, Alzheimer's disease, head injury, stroke and multiple sclerosis maybe carried out by the infusion of neurotrophic factors (e.g. Glial cell derived neurotrophic factor (GDNF)) to protect and repair failing or damaged nerve cells. Neurotrophins may also be infused to support neural grafts transplanted into damaged or malfunctioning areas of the brain in order to restore function.

A number of neurosurgical catheters have been developed previously that can be guided (e.g. using a stereo guide) to desired target sites within the brain parenchyma. For example, it has been described previously in WO2003/077785 how a fine neurosurgical catheter formed from carbothane can be inserted into the brain using a guide tube arrangement of the type described in U.S. Pat. No. 6,609,020. In one embodiment described in WO2003/077785, a guide tube is inserted into the brain along a guide wire using a stereotactic placement technique. This allows the distal end of the guide tube to be accurately located just short of the desired brain target. A fine neurosurgical catheter, reinforced by a fine tungsten guide wire, is then inserted into the implanted guide tube and passed along the guide tube until it reaches the distal end thereof. The catheter tip then exits the guide tube and catheter insertion is continued until the catheter tip reaches the desired target. The fine guide wire is then withdrawn from the catheter lumen leaving the catheter in situ. The use of fused silica catheters for the delivery of drugs in to the brain parenchyma has also been proposed previously. Fused silica catheters are, however, relatively brittle and tend to fracture if excessively bent. This makes such catheters unsuitable for long term implantation within a subject.

It would be advantageous to have a catheter that is stiff enough to allow it to be inserted into a target site within the brain, without the need for a guide wire.

### SUMMARY OF THE INVENTION

The invention provides the use of zirconia or aluminia for medical purposes, especially for the production of medical devices, especially neurosurgical devices. In particular, the invention provides the use of zirconia or alumina tubing for such purposes. The invention also provides neurosurgical tubing, especially catheters and guide tubes that comprise zirconia or aluminia, especially rigid forms of those ceramics, and particularly rigid tubing made from those ceramics. Zirconia and alumina may form or be used in conjunction with such devices described in WO03/077784 and U.S. Pat. No. 6,609,020, both of which are incorporated by reference. The ceramic used is preferably zirconia.

The invention provides the use of a stiff or rigid zirconia or alumina tube as an MR & CT compatible tube, especially a guide tube to facilitate the implantation of a neurological instrument. Such a guide tube may be implanted just short of a desired target. Following implantation, a catheter is threaded through the guide tube's bore. Upon completion of the surgical procedure the guide tube and catheter may be removed.

In an alternative embodiment, the zirconia or alumina tube may be a stiff or rigid MR & CT compatible catheter, for delivery to a target, especially a target within the brain. Such a catheter may be used with a stereotactic system. The tube may be used to deliver therapeutics to the target site.

According to a first aspect of the present invention, there is provided a delivery or sampling device for insertion into a subject. The delivery or sampling device is preferably a catheter comprising a tube, substantially formed from, or comprising a rigid layer substantially formed from, zirconium dioxide or aluminium oxide.

The catheter is preferably a neurosurgical catheter, for insertion into the brain parenchyma of a subject. The catheter comprises a length of stiff tubing, the tip of which can be accurately located at a required target point or region within the brain. The catheter may comprise one or more lumens as required and, when implanted, may delivery any type of therapeutic agent or fluid directly to a target region within the brain.

A rigid catheter in accordance with the present invention has the advantage that it can be accurately guided to a target site within the brain parenchyma. In particular, the catheter will not be significantly deflected from the required insertion direction even when passed through virgin brain tissue or into tough matter such as brain tumours or similar tissues. A catheter, of the present invention thus has the advantage of not requiring any additional reinforcement (e.g. using a stiffening wire or cannula) during implantation.

A catheter of the present invention is particularly suited for use in combination with guide tube devices such as those described in WO2003/077785 and U.S. Pat. No. 6,609,020. As mentioned above, WO2003/077785 describes how a guide tube can be stereotactically implanted in the brain so that its distal end is just short of a desired target. A fine flexible catheter, reinforced by an even finer tungsten wire, is then inserted into the brain parenchyma through the guide tube. During catheter insertion, the catheter tip exits the distal end of the guide tube and is forced a short distance through virgin brain tissue to the desired target. It has, however, been found that in some instances the tip of the catheter described in WO2003/077785 can still deviate from the axis of insertion defined by the longitudinal axis of the guide tube during such an implantation process. Even relatively small deviations from the identified target site are undesirable as they can significantly reduce treatment efficacy and may cause unwanted damage to sensitive regions of the brain. These deviations from the required target have been found to be a particular problem when the catheter has a small outside diameter (thereby requiring the use of a very thin tungsten wire) and/or when the tip has to be inserted into relatively tough tissue (such as a brain tumour or similar tissue). The removal of the tungsten guide wire after catheter implantation without disturbing catheter placement can also prove problematical. The present invention, through the provision of the stiff catheter, overcomes the need to use a reinforcing guide wire during catheter implantation whilst also allowing accurate guiding of the catheter tip to the required target. The present invention thus avoids certain problems that can arise when using catheters of the type described in WO2003/077785.

Alternatively, the catheter of the invention may be used without a guide tube, it being stiff enough to penetrate brain tissue without deviating from the desired axis of insertion. Accordingly, the device may be located stereotactically, using a stereotactic guide or other interface to direct the positioning of the device.

The catheter tube comprises a rigid layer of a solid ceramic, specifically zirconium dioxide or aluminium oxide. The rigid layer is preferably formed substantially from that ceramic, the layer comprising at least 95% by weight of the ceramic, preferably at least 97% by weight, more preferably at least 99% by weight, more preferably 100% by weight.

Zirconium dioxide has been used in prior art devices, such as catheters described in EP1136085. In that application, zirconium dioxide was combined with a plastic material to provide a strengthened and radio-opaque wall. Alternatively, other prior art devices have used networks of braided ceramic fibres, as described in US20050163954, such catheters being strengthened by the braided fibres, but still remaining flexible.

The catheter of the present invention is rigid, unlike the prior art catheters. Rigidity is provided by the layer of ceramic in the catheter wall. The catheter wall may also comprise other layers, for example, the catheter may be a tube that is coated with the ceramic layer. In that case, the tube may be made of a flexible material, a rigid material or a composite material with flexible and rigid characteristics, such as coated fused silica.

The ceramic layer preferably covers at least 75%, more preferably at least 80%, more preferably at least 85%, more preferably at least 90%, even more preferably 100% of circumference of the catheter tube.

The ceramic layer is preferably substantially solid. If openings, holes or apertures are provided in the layer, they are preferably in fluid connection with the lumen of the catheter. The catheter may comprise a single fluid aperture at its distal end and/or one or more apertures may be provided in the sides of the catheter.

The catheter is preferably of an appropriate size for neurosurgical implantation. For example, the outer diameter of the catheter is preferably between 100 μm and 1.5 mm, more preferably between 200 μm and 1.25 mm, more preferably between 200 μm and 500 μm, preferably between 220 μm and 280 μm, more preferably between 230 μm and 250 μm.

The inner diameter of the catheter is preferably between 70 μm and 250 μm, preferably between 80 μm and 120 μm, more preferably between 90 μm and 110 μm.

The walls of the catheter may be coated to improve elution, or to reduce friction when the catheter is inserted or removed.

The tip of the catheter at its distal end may be shaped to improve deliver of fluids and to reduce trauma when the catheter is inserted. For example, the tip may be rounded in shape at its end. Also, the end of the catheter may include a series of steps, reducing the outer diameter of the catheter in the region of the tip.

The interior or exterior walls, especially the exterior walls, may be shaped or profiled, for example provided with steps or grooves around the circumference of the wall or longitudinally, along the length of the catheter. For example, the outer diameter of the catheter may be reduced towards the tip using one or more steps or by tapering the walls. Such profiling or shaping may be used to promote or discourage fluid movement along the catheter walls. The walls of the catheter may also be provided with markings to indicate how far the catheter has been inserted into a patient.

In certain embodiments, the rigid tube of the catheter may be connected to a flexible tube at the proximal end of rigid tube. This may aid in connecting the catheter to a supply device such as a hub or port. Alternatively, the catheter may be connected directly to a supply tube from that supply device.

For a long-term implantable embodiment, the proximal end of the catheter may be connected to a supply tube. The supply tube may be flexible and may have an outside diameter that is greater than the flexible tube emanating from the proximal end of the catheter. The connection between the flexible tube and the supply tube is conveniently located outside of the brain parenchyma and is preferably located outside the skull. Advantageously, fixing means are provided for securing the flexible tube of the catheter in place (e.g. by fixing it to the skull) after implantation; this ensures that the catheter tip does not deviate from the desired position within the brain parenchyma. The supply tube may, for example, be connected to the flexible tube by a connector or hub that is secured (e.g. screwed) to the outside of the skull and subcutaneously buried under the scalp.

The catheter may be designed for long term implantation and is thus preferably fabricated from materials that are suitable for long term implantation.

For a non-implantable embodiment, the supply tube & the flexible tube can be replaced by one continuous element, which may be connected to the rigid tubing as required. This may be preferable as it is not always desirable to leave a rigid device implanted within the skull. In such an embodiment, the supply tube may be connected prior or after insertion of the catheter. For example, the catheter may be inserted stereotactically either with or without a guide tube. If necessary, its position may be maintained by an external clamp. The proximal end of the catheter may then be temporarily connected to a supply tube connecting it to a delivery hub or pump.

It should also be noted that the catheter is preferably passively insertable (i.e. it is preferably not actively steerable).

The present invention may also comprise a neurosurgical kit comprising; a neurosurgical catheter as described above and a neurosurgical guide tube device, wherein the neurosurgical guide tube device comprises a guide channel (e.g. formed by an elongate guide tube) through which the neurosurgical catheter can be passed. The neurosurgical guide tube device is preferably of the type described previously in U.S. Pat. No. 6,609,020 or WO2003/077785.

Conveniently, the outer diameter of the catheter is less than the internal diameter of the guide channel and such relative diameters are preferably arranged so that the catheter fits snugly within the guide channel. The guide channel of the guide tube thus acts to guide the catheter to the desired target even after the distal end of the tip has exited the guide channel. Based on the teachings contained herein, a skilled person would thus be able to select the relative lengths of the catheter and the guide tube for the particular surgical procedure being performed; this selection would vary from subject to subject and would take into account the required proximity of the guide tube to the desired target and the depth of the target within the brain. It should also be noted that the guide tube and/or catheter may be manufactured as standard lengths and tailored (e.g. cut by the surgeon) to the required length before or during the surgical procedure. The kit may also include other components. For example, a subcutaneous drug delivery pump and/or additional fluid tubing may be provided. A stereoguide for implanting the guide tube device may also be provided as part of the kit.

As described above, one of the primary uses of the delivery or sampling device of the invention is as a neurosurgical catheter. Also envisaged is the use of the device as a biopsy needle. In that case, the device preferably comprises a rigid tube formed from zirconium dioxide or aluminium oxide or comprising a rigid layer of such a ceramic, the tube being appropriately shaped for use as a biopsy needle. For example, the tip of the tube may be shaped to form a point.

Alternatively, the device may be used for the delivery of a solid agent, such as a pellet of a radio isotope. In that case, the device may be formed as a rigid rod or tube made from or comprising a rigid layer of zirconium dioxide or aluminium oxide. The rod or tube may be shaped to allow the solid agent to be mounted upon it or delivered through it.

Also, the device may be used to deliver an electrode to a site of interest. Accordingly, the device may be formed as a rigid rod or tube made from or comprising a rigid layer of zirconium dioxide or aluminium oxide and comprising an electrically conducting material extending along the length of the tube or rod and being exposed or electrically connected to an exposed area on the surface of the rod or tube. The rod or tube is preferably arranged to allow connection of the electrically conducting material to an electrical supply.

Also provided by the invention is a rigid implantable device, such as a bone implant, formed from or including a ceramic, especially zirconium dioxide or aluminium oxide.

The devices of the invention are preferably formed from, or comprise zirconium oxide.

According to another aspect of the invention, a method of manufacturing a device comprises the steps of extruding a rigid tube or rod of zirconium dioxide or aluminium oxide or coating a tube or rod with a rigid layer of zirconium dioxide or aluminium oxide (e.g. a flame-deposited ceramic coating).

Aspects of the shaping, profiling, marking and sizing, as discussed above in relation to the catheter may also be found on other devices according to the invention.

According to a fifth aspect of the invention, a method of delivering a therapeutic substance to a target with the brain parenchyma of a subject is provided. The method comprises the steps of (i) taking a delivery device, especially a catheter, according to the invention and (ii) inserting the device into a subject, especially into the brain parenchyma of the subject.

Advantageously, step (ii) comprises inserting the catheter into the brain parenchyma through a previously implanted guide tube device. An initial step may thus be performed of implanting a guide tube device, such as a guide tube device of the type described previously in U.S. Pat. No. 6,609,020 or WO2003/077785, in the brain parenchyma of a subject. During the implantation of the guide tube device, its distal end maybe located (just) short of the required target within the brain parenchyma. Advantageously, step (ii) comprises passing the catheter through the previously implanted guide tube device until the tip of the catheter reaches the desired target within the brain parenchyma. Conveniently, the tip may be guided with the aid of the guide tube device as it exits therefrom and is moved towards the target.

Once implanted, the step (iii) maybe performed of delivering a therapeutic substance to the brain parenchyma via the implanted catheter. A catheter may be implanted whenever delivery of a therapeutic substance is required or it may advantageously remain implanted for the long term (e.g. for months or years).

When using a device according to the invention, or other implantable devices, it may be advantageous to be able to advance or retract the device without the surgeon manually pulling or pushing on the device. This is particularly important where the surgeon may be acting remotely. Accordingly, there is provided an implantable device, such as a catheter or a guide tube, comprising an advancement means for retracting or advancing a portion of the device along an axis of insertion into a subject, minimising tissue trauma. The means may advance or retract the device using any appropriate method, examples being a slide, a piezo-electric motor or a helical screw, such that when the means is turned, the device is advanced or retracted. The means may be used to advance or retract the device over a length appropriate to the device's use, but is preferably only used to advance or retract the device short distances, such as less than 10 mm. One or both of the device and the advancement means may be provided with a scale to indicate how far the device has been advanced or retracted. In addition, one or both of the device and the advancement means may be provided with a stop to prevent movement beyond a certain maximum position. Such a stop may be moveable prior to use of the advancement means and then fixable in the desired position.

Another aspect of the present invention provides an optical instrument for use in surgery, the instrument comprising a tube having at least one optical fibre arranged within a bore region, a wall of the tube comprising a rigid layer formed substantially from a ceramic selected from zirconium dioxide or aluminium oxide. Preferably, the at least one optical fibre comprises a plurality of optical fibres. It is also preferable that the at least one optical fibre extends between a distal end of the tube and a proximal end of the tube and is capable of transporting light between both ends. Embodiments of the optical instrument advantageously provide a rigid tube that is capable of transporting light into, and/or out of, a patient. Due to the rigid nature of the tube it can be accurately guided to a target site within the patient and will resist deflection by internal parts of the patient's body, such as, brain tissue or brain tumours.

Preferably, the at least one optical fibre is further arranged to receive light from outside the tube at the distal end, and provide the received light at the proximal end to an image reproducing means for reproduction of an image present at the distal end. It is also preferable that at the distal end of the tube the at least one optical fibre is terminated with a substantially convex profile so that the instrument's field of view is large with respect to an outer diameter of the tube. These embodiments are capable of being inserted inside a patient in order to collect images therefrom. Further, the area imaged by these embodiments can be large in comparison to the area of the opening at the distal end of the tube through-which images are collected. Preferably, the optical instrument is further arranged to be coupled to a light source for delivering light to the distal end. It is an advantage of this embodiment that bright and detailed images can be obtained.

Preferably, the at least one optical fibre is further arranged to emit light out of the tube from the distal end. It is also preferable that the at least one optical fibre is terminated at the distal end with a profile for causing at least one of the following effects in the emitted light:

a. diffusion which is wide with respect to an outer diameter of the tube;
b. focusing which is narrow with respect to an outer diameter of the tube; and
c. refraction at 90° to a central axis of the tube.

It is additionally preferable that the emitted light is received from a light source coupled to the proximal end. It is an advantage of these embodiments that the optical instrument can be used to deliver light to a target site inside a patient, for example, to effect treatment of an illness. It is a further advantage that the way in which light is delivered to the patient from the optical instrument can be adjusted in dependence on the type of treatment to be administered.

A further aspect of the present invention provides a surgical probe comprising a tube terminated at a distal end by a tip, at least a wall of the tube comprising a rigid layer formed substantially from a ceramic selected from zirconium dioxide or aluminium oxide, the probe further comprising a first electrode housed within a bore region of the tube and positioned towards the distal end. Preferably, the first electrode is a disc electrode which is coaxial with the tube, positioned adjacent to the tip and in electrical communication with a proximal end of the tube. These embodiments advantageously provide a rigid mono-polar probe which is suitable for insertion inside a patient and for measuring the electrical impedance of internal parts of the patient's body. Due to the rigid nature of the probe it can be accurately guided to a target site within the patient and will resist deflection by internal parts of the patient, such as, brain tissue or brain tumours.

Preferably the surgical probe further comprises a second electrode housed within the bore region, positioned the proximal side of the first electrode, wherein both electrodes are electrically insulated from each other. It is additionally preferable that the second electrode is a disc electrode which is coaxial with the tube, positioned adjacent to the first electrode, and in electrical communication with the proximal end. These embodiments advantageously provide a rigid bi-polar version of the surgical probe.

### BRIEF DESCRIPTION OF THE DRAWINGS

The invention will now be described, by way of example only, with reference to the accompanying drawings in which;

FIG. 1 illustrates a prior art neurosurgical catheter and guide tube arrangement,

FIG. 2 illustrates a catheter of the present invention, and

FIG. 3 illustrates a catheter of the present invention inserted into an implanted guide tube.

FIG. 4 illustrates an advancement means according to the present invention, on a rigid guide tube of the present invention (A) and on a catheter (B) of the present invention.

FIG. 5 shows the advancement means of the present invention.

FIG. 6 is an exploded view of the advancement means.

FIGS. 7 and 8 illustrate the optical instrument of the invention.

FIGS. 9 and 10 illustrate the surgical probe of the invention.

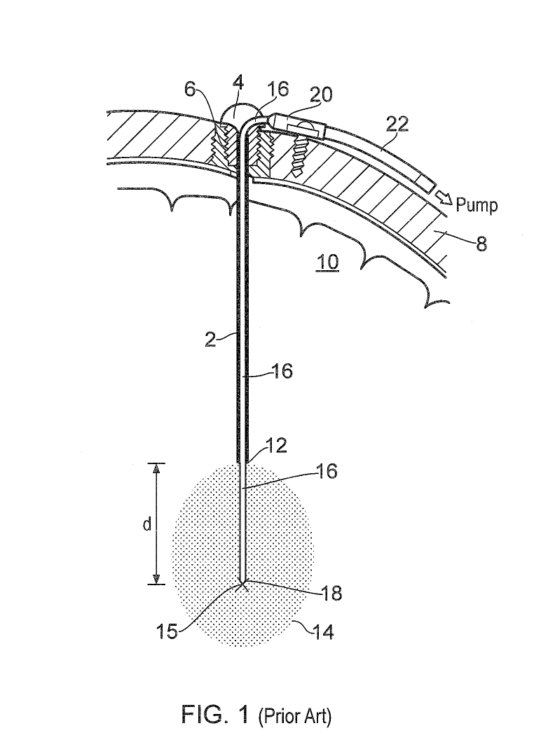

Referring to FIG. 1, a prior art implanted fluid delivery system of the type described in WO2003/077785 is illustrated. The fluid delivery system comprises a guide tube device comprising an elongate guide tube 2 having a head portion 4 at its proximal end. The head portion 4 has an external thread 6 to allow attachment to a burr hole formed in the skull bone 8 of a subject. The guide tube device is inserted stereotactically into the brain parenchyma 10 using a stereoguide device. In particular, the guide tube device can be accurately inserted in the brain along a predefined axis of insertion such that its distal end 12 is located just short (by a distance d) of a target point 15. More details concerning accurate (e.g. stereotactic) insertion of the guide tube can be found elsewhere; for example, see WO2003/077784, WO2003/077785 and U.S. Pat. No. 6,609,020.

After the guide device has been implanted, a catheter is inserted through the head portion 4 and into the guide tube 2. The catheter comprises a length of flexible fine tubing 16. The tubing has an outside diameter of 1 mm or less. During implantation, the fine tubing 16 is inserted into the guide tube 2 and advanced therethrough until the distal end 18 of the fine tube 16 protrudes a distance “d” from the distal end 12 of the guide tube 2 and thereby reaches the target point 15.

As described in WO2003/077785, when a flexible catheter is used, it is typically reinforced by a guide wire (not shown) during implantation to prevent the catheter significantly deviating from the required axis of insertion as it exits the distal end 12 of the guide tube 2 and is driven towards target point 15. Once implanted, the guide wire is withdrawn from the catheter leaving the catheter in situ.

The fine tube 16 of the catheter is connected to a hub 20 that is screwed to the outside of the skull 8. A supply tube 22 is in fluid communication with the fine tube 16 via a channel formed in the hub 20. The supply tube 22 may receive fluid from an implanted drug pump, the fluid then being routed along the fine tube 16 to the target volume 14. The catheter and guide tube device are arranged to be long term implantable thereby allowing drug delivery, either continuously or intermittently, over prolonged periods of time.

Although the prior art neurosurgical catheter system described above with reference to FIG. 1 typically enables accurate catheter placement, it has been found by the present inventors that it can sometimes suffer from a number of problems. For example, the use of a fine tube 16 (e.g. having an outer diameter of 1 mm or less) means that only a relatively small diameter guide wire can be used to stiffen the catheter during insertion. This means that the distal end 18 of the catheter can still wander off course during implantation, especially when insertion into tough tissue (such a brain tumour or cyst) is required. It has also been found that the process of removing a fine guide wire from the fine tubing 16 can prove difficult to perform in a surgical environment and in particular that the process of guide wire removal can sometimes reduce the accuracy with which the distal end 18 is located relative to the target point 15.

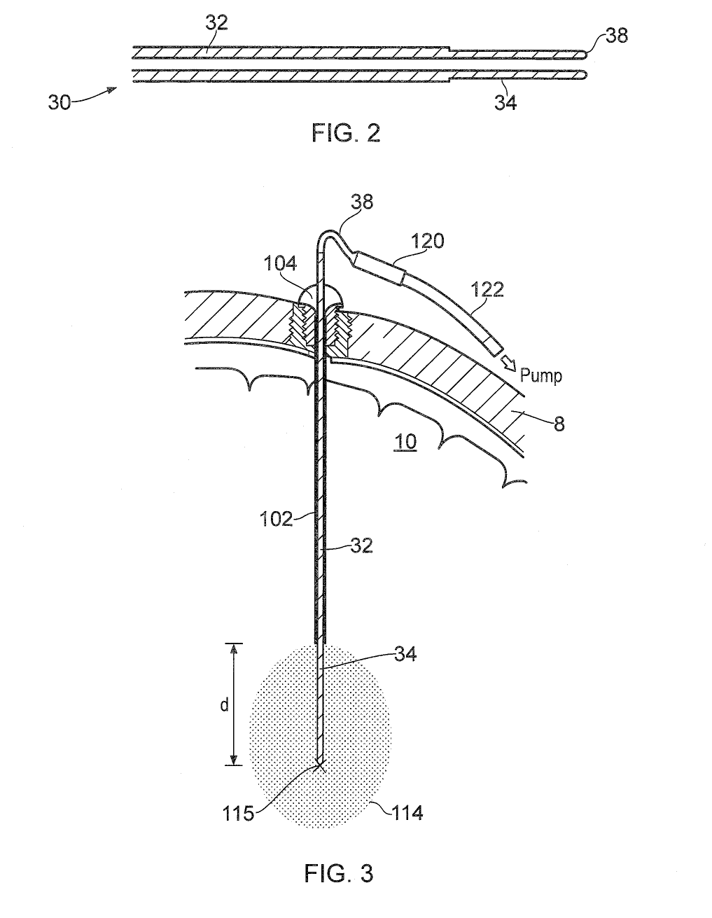

Referring to FIG. 2, an improved catheter 30 according to the present invention is shown.

The catheter 30 comprises a length of rigid tube 32. The tube is formed from or comprises a layer of a rigid ceramic, especially zirconium dioxide and has an outside diameter of around 1 mm or less.

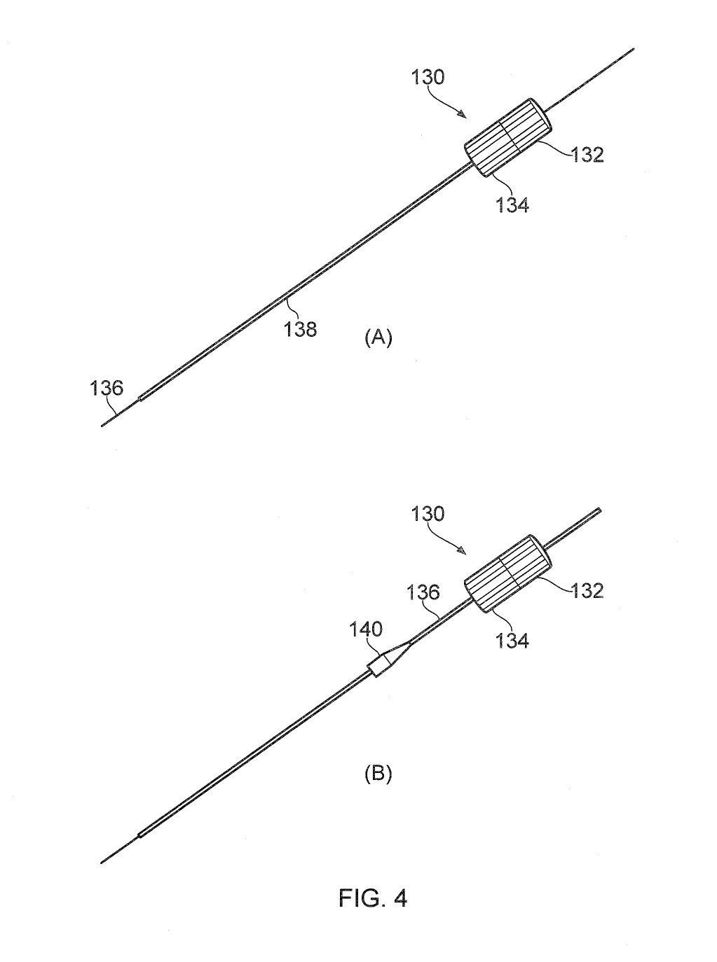

The tip 34 of the catheter is shaped. The distal edge of the catheter is rounded to reduce trauma on insertion. The external wall is provided with a series of steps 38 to gradually reduce the external diameter of the catheter. The external wall may also be provided with grooves or channels (not shown). The catheter may be coated with, for example, polyimide. The catheter may be provided with an advancement means 36 to allow automatic advancement or retraction of the catheter. The advancement means is shown in more details in FIGS. 4 to 6. The external surface of the catheter is provided with a scale to indicate how far the catheter has been moved. Such an advancement means could be used with any other catheter or implantable device that is to be advanced or retracted along an axis.

In this embodiment of the invention, a single lumen is provided through the catheter and that fluid will exit the catheter through a single aperture located at the distal end of the tube 32. It should, however, be noted that multiple lumen variants of the catheter maybe provided. Furthermore, the fluid aperture may be located in a different position to that shown in FIG. 2; for example, an aperture may be provided on the side of the tube. If necessary, more than one fluid aperture may also be provided.

In other embodiments, not shown, the device of the invention comprises a rigid rod, needle or implant, formed from or comprising a rigid ceramic especially zirconium dioxide or aluminium oxide.

The catheter 30 can be fabricated using any one of a number of techniques. In a preferred embodiment, the catheter 30 is fabricated by coating a long fused silica tube with the required ceramic. Alternatively, the ceramic may be extruded to form the rigid tube 32 and then sintered. Other devices of the invention may be fabricated in similar manners, either by coating a support such as a rod with the ceramic, or by extruding the ceramic to form the device.

Referring to FIG. 3, implantation of a catheter of the present invention in a subject will be described. In common with prior art arrangements of the type described with reference to FIG. 1, a guide tube device comprising a guide tube 102 and a head portion 104 is firstly implanted in a subject (e.g. a person or an animal) using known stereotactic techniques. The guide tube 102 may thus define the axis of insertion to a target point 115 for delivery of a therapeutic agent to a target volume 114 within the brain parenchyma 10. A thread 106 provided on the head portion 104 firmly anchors the guide device to the skull bone 8 of the subject. The catheter 30 of the present invention is inserted into the guide tube 102 through the head portion 104. The tip 34 of the catheter is then fed along the guide tube 102 towards the target volume 114. The catheter 30 is inserted into the guide device until the distal end of the catheter tip 34 extends a distance d from the distal end of the guide tube 102. This distance d can be set by providing a mark or other indicator (e.g. a graticule or scale) on the rigid tube 32 and a corresponding mark on the head portion 104; alignment of these marks indicates that the distal end of the catheter has advanced the required distance d from the distal end of the guide tube 102. Imaging techniques may also or alternatively be used during implantation to identify catheter tip position.

The guide tube 102 is arranged to have an internal diameter that is only slightly larger than the outside diameter of the rigid tube 32 of the catheter. In this manner, the stiff tube 32 is guided along the axis of insertion defined by the guide tube 102 and, importantly, such guidance is still provided even when the distal end of the catheter 30 exits the guide tube 102. The inherent stiffness of the catheter thus accurately guides the tip to the target point 115 without the need to use any kind of wire or cannula to reinforce the catheter. The problems associated with using, and removing, a guide wire are thus mitigated thereby making the catheter implantation process simpler and quicker whilst providing high targeting accuracy. Furthermore, a catheter of the present invention can be primed before insertion thereby preventing the introduction of air in to the brain. In order to allow the connection of the catheter to a hub or other device, the catheter may be connected to a flexible tube 38. Once the distal end of the catheter 30 has been placed at the target point 115, the flexible tube 38 can be bent either within or above the head portion 104 of the guide device. The flexible tube is sufficiently bendable to be routed (without fracturing) through a right angle in the vicinity of the skull bone (e.g. within the head portion 104 of the guide tube device) to allow subcutaneous burying of the catheter. It should be noted that it is the flexible tube 38 that is bent and there is no need to bend the stiff tube 32.

In the present embodiment, the proximal end of the flexible tube 38 is attached to a hub 120 that may be screwed to the skull bone 8 of the patient thereby securing the catheter in place if the catheter is for long term implantation or may be clamped above the head, especially if the catheter is only for short term implantation. A supply tube 122 for supplying fluid from an associated (e.g. implanted) drug pump is also connected to the flexible tube 38 via the hub 120. It should, however, be noted that the hub 120 and supply tube 122 are not essential parts of the invention and merely provide a convenient means for routing fluid to the catheter for onward delivery to the target volume 114. The proximal end of the rigid tube could be connected, permanently or whenever required, to any (e.g. implanted or external) fluid source when fluid delivery through the catheter is required. The length of the flexible tube 38 and/or tube 122 may thus be selected to permit the required fluid connections.

It should also be noted that the catheter of the present invention can also allow the distance d between the distal end 112 of the guide tube 102 and the required target point 115 to be increased if required without significantly degrading targeting accuracy. Increasing this distance can reduce the amount of damage to brain tissue and can also reduce fluid reflux along the interface between the brain tissue and the guide tube. The tip length and/or the distance d between the distal end 112 of the guide tube 102 and the target point 115 can thus be varied as required on a patient-to-patient basis to provide the optimum treatment regimen.

It is also important to note that the catheter of the present invention can be used with a different type of guide tube than that described above and may even be used without any kind of guide tube device. For example, a catheter or other appropriate device of the present invention may be inserted directly into the brain parenchyma, without any guide tube. In such an instance, the device, especially a catheter, is inserted stereotactically into the brain parenchyma using a stereoguide device. In particular, the device can be accurately inserted in the brain along a predefined axis of insertion such that its distal end is located at a target point. Details of stereotactic implantation of devices are described in WO2003/077784, WO2003/077785 and U.S. Pat. No. 6,609,020, which are incorporated by reference herein.

Also described above is the implantation of a device that comprises a flexible tube for connection of the device to a supply device. A device according to the invention may not comprise such a flexible tube and may simply comprise a rigid portion of tubing. The tubing may be directly connectable to a supply device, if needed.

It should also be noted that although the above examples refer to delivery of therapeutic agents (e.g. drugs, viruses etc) through the catheter, it would also be possible to collect a fluid using the catheter. The above described catheter is particularly suited for use in neurosurgical applications where catheter insertion directly into the brain parenchyma through a hole in the skull is required. The catheter can, however, also be used for other medical applications. For example, it may be used in applications where fluid needs to be delivered to an accurately defined target within an organ (e.g. to the liver, kidneys etc). The skilled person would thus be aware of the numerous applications for the catheter described herein.

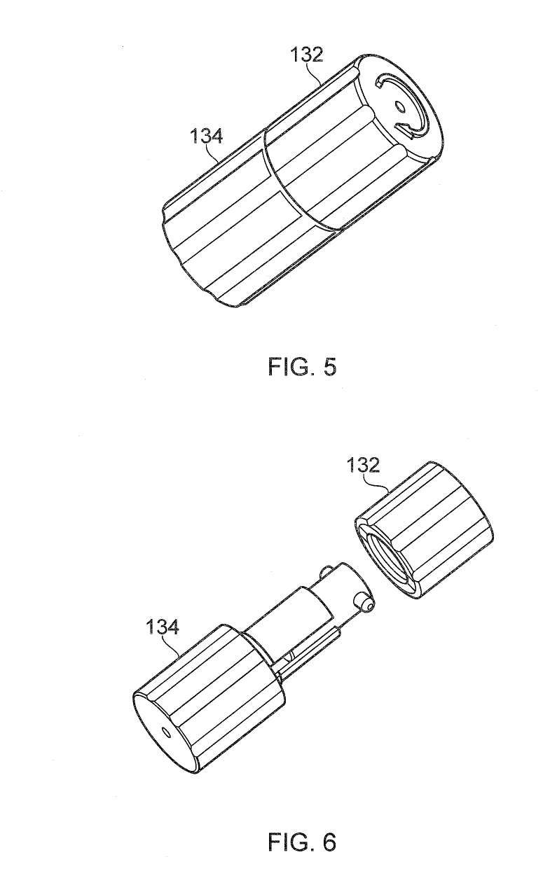

Referring now to FIGS. 4 to 6, an implantable device may be provided with an advancement means. As shown in FIG. 4, the advancement means 130 comprises a controller 132 and an actuator means 134. Activation, in this case turning, of the controller results in advancement or retraction of the instrument to which the advancement means is attached. As shown in FIG. 6, the actuator means may be a linear actuator which translates rotational movement of the controller. Alternatively, the actuator means may be another mechanical, electromechanical or piezoelectric actuator. A variety of controllers may also be used.

As shown in FIG. 4, the advancement means may control a catheter 136 or infusion tube within a guide tube 138. Alternatively the catheter or tube may be used without the guide tube. In the latter case, the catheter or tube may be provided with an endstop 140, to prevent further advancement (or retraction) of the catheter.

A device bearing the advancement means may be implanted into a subject's brain, using stereotactic techniques as described previously. The device may be implanted such that the tip of the device is short of the target site. The device, or a portion of the device may then be advanced using the advancement means such that it reaches the target site. For example, the device may comprise a guide tube that is inserted short of the target site. The device may further comprise a fine infusion tube within the guide tube. The advancement means may then be used to advance the infusion tube out of the guide tube and towards the target site, until the tip of the infusion tube reaches the target. The advancement means may also be used to retract the device away from the target. This may be done to remove the device. The device might also be advanced or retracted during infusion of an agent to increase the target area.

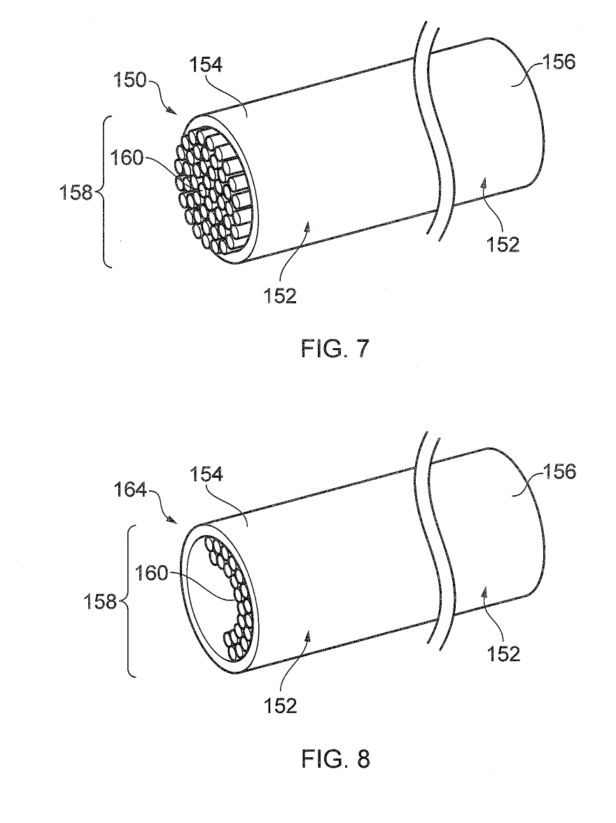

Referring now to FIG. 7, an optical instrument 150 is shown comprising a hollow cylindrical tube 152 having a distal end 154 and a proximal end 156. A central bore of the tube 152 houses a cylindrical fibre optic bundle 158 comprising a plurality of fibre optic strands 160. Each of the fibre optic strands 160 extends between the distal end 154 and the proximal end 156 and is capable of transporting light between both ends. Each of the fibre optic strands 160 is terminated at the distal end so that the bundle 158 terminates with a substantially convex profile. More specifically, a radially outermost layer of fibre optic strands 160 of the bundle 158 terminate such that they are flush with the end of the distal portion 154. A layer of fibre optic strands 160 which are immediately adjacent to, and radially inward of the radially outermost layer extend just beyond the radially outermost layer. Each subsequent radially inner layer of fibre optic strands 160 extends just beyond an immediately adjacent and radially outer layer of fibre optic strands 160. Accordingly, fibre optic strands 160 which are at a centre of the bundle 158 extend the greatest distance beyond the distal end 154.

The proximal end 156 of the optical instrument 150 is arranged to be coupled to an image reproducing means (not shown). The image reproducing means is capable of reproducing an image from light received at the distal end 154 and transported by the bundle 158 to the proximal end 156. Accordingly, the image reproducing means when combined with the optical instrument 150 is capable of reproducing an image present at the distal end 154 of the optical instrument 150. Additional lighting means (not shown) can also be provided to illuminate the area at the distal end 154 and thereby increase the quantity of light received by the optical instrument 150 and the quality of the image provided by the image reproducing means, as is well known in the art. Suitable image reproducing means will be apparent to the skilled person and include, for example, an eye piece or a charge coupled device (CCD) camera. Also, suitable methods of coupling the tube 152 and the bundle 158 to the image reproducing means will be apparent to the skilled person and are outside the scope of the present embodiment.

The profile with which the bundle 158 terminates at the distal end 154 defines how the image present at the distal end 154 is transmitted and refracted before it is transported to the proximal end 156. Moreover, the termination of the bundle 158 at the distal end 154 acts as a lens between the image at the distal end 154 and the proximal end 156. Further, the profile of the termination defines the properties of the lens, i.e. how the image at the distal end 154 is transmitted and refracted before it is provided at the proximal end 156. As discussed above, the profile in FIG. 7 is substantially convex and therefore, it acts as a substantially ‘fish-eye’ shape lens. An advantage of a convex profile is that it provides a large field of view with respect to an outer diameter of the tube 152. A convex profile also distorts the image in order to provide a large field of view however, the distortion can be compensated for in order to provide an image having a large field of view and minimal distortion. For example, the optical instrument 150 together with an image reproducing means can be used to view, a calibrated artifact in order to obtain an optical error-map. The optical error-map allows any subsequent image provided by the arrangement to be mapped and real-time corrected, as is well known in the art. Accordingly, the arrangement is capable of providing highly anaclastic optical performance with a large field of view from the relatively narrow diameter tube 154.

The optical instrument 150 is suitable for being inserted inside a patient as part of a surgical procedure. For example, the optical instrument 150 can be inserted through a patient's skull and inside the patient's brain as part of a neurosurgical procedure. The optical instrument 150 is particularly well suited to neurosurgical applications by virtue of its construction. More specifically, the tube 152 comprises a rigid layer formed substantially from a ceramic selected from zirconium dioxide or aluminium oxide which gives the tube 154 a rigid material property. A rigid instrument is advantageous because the instrument can be accurately guided to a target site within the brain parenchyma. In particular, the instrument will not be significantly deflected from the required insertion direction even when passed through virgin brain tissue or into tough matter such as brain tumours or similar tissues. The instrument therefore has the further advantage of not requiring any additional reinforcement during insertion. Additionally, the optical instrument 150 is suitable for operating within a magnetic resonance environment by virtue of the fact it is not constructed from materials which are influenced by a magnetic field.

Various modifications can be made to the embodiment of FIG. 7, such as, for example, rather than having an independent light source the optical instrument itself can be provided with a light source which is capable of providing illumination to an image located at the distal end 154.

Thus far the optical instrument of FIG. 7 has been described for use as an endoscope, and in particular, a neuro-endoscope. However, it is within the scope of appended claims that the optical instrument of FIG. 7 is suitable for use as a fibre-optic delivery instrument.

In order to function as a fibre-optic delivery instrument, the proximal end 156 is coupled to a light source (not shown) according to a method which will be apparent to the skilled person and therefore is outside the scope of the appended claims. According to this arrangement, light from the light source is received at the proximal end 156 by the bundle 158 and is transported to the distal end 154 via the bundle 158. On reaching the end of the bundle 158 at the distal end 154 the light is emitted out of, and away from, the optical instrument 150. As discussed above, the termination of the bundle 158 acts as a lens which transmits and refracts the light according to the profile of the termination. However, in contrast to the above, the light is emitted from the distal end 154 rather than received at the distal end 154. The bundle 158 terminates with a convex profile and therefore, light is emitted from the distal end 154 with a wide-angle of dispersion. Such an arrangement is particularly suitable for surgical procedures, such as, for example, photodynamic therapy (PDT) wherein it is desirable to have light dispersed over a wide area to improve treatment effectiveness.

As seen more particularly on FIG. 8, an alternative fibre-optic delivery tube 164 comprises the bundle 158 terminating with a substantially concave profile at the distal end 154. According to the embodiment of FIG. 8, light is emitted from the distal end 154 with a narrow angle of dispersion and therefore, the light emitted from the distal end 154 is focused on a particular point or region. Further, the area of the point or region is sized and shaped in dependence on the precise shape of the concave profiling and therefore, the area of the point or region can be changed by altering the shape of the concave profile. A concave profile is particularly suitable for surgical procedures, such as, for example, tissue ablation, wherein it is desirable to have a highly focussed beam of light which can be directed towards a predefined area to administer treatment most effectively.

It is also within the scope of the appended claims that the bundle is terminated with any profile other than a convex or concave profile. Moreover, the profile could be shaped to generate a particular effect in the light either entering or exiting the distal end. For example, when the optical instrument is used as a fibre-optic delivery instrument, it is often desirable for light exiting the distal end to be emitted substantially perpendicularly to the axis of the tube of the optical instrument. Such an arrangement is particularly advantageous in some PDT or tissue ablation applications, wherein the instrument can be rotated and/or moved axially once it has been located in position within a patient to project light onto tissue which is radially outward from the distal end.

Additionally, it is within the scope of the appended claims that the outer surface of the optical instrument can be encoded with an absolute scale so that the instrument's axial position inside a patient can be quickly and easily determined. Accordingly, the optical instrument can be reliably guided to the correct depth within a patient.

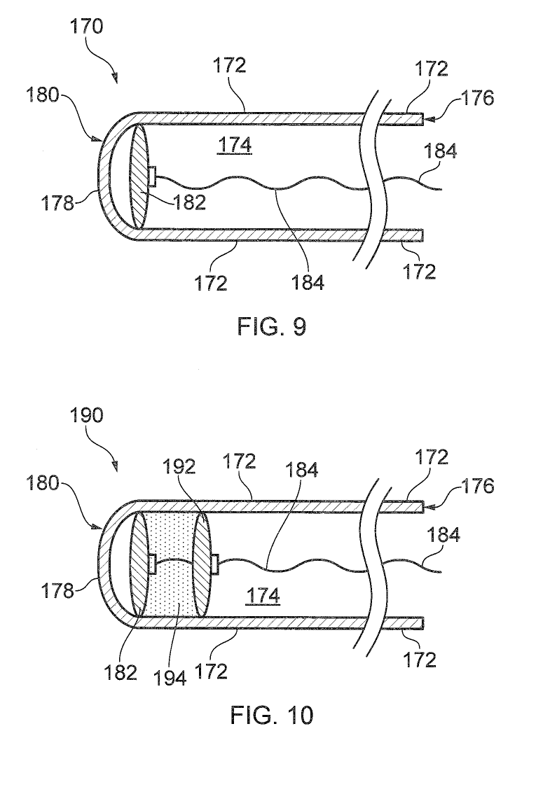

Referring now to FIG. 9, wherein a cross section of a surgical probe 170 is shown. The probe 170 comprises a hollow cylindrical tube 172 having a central bore region 174. The tube 172 is open at a proximal end 176 and is terminated by a hemispherical tip 178 at a distal end 180. A disc electrode 182 is positioned inside the bore region 174 and towards the distal end 180. Preferably, the disc electrode 182 is coaxial with the tube 172 and is positioned immediately behind the tip 178. The disc electrode 182 is conducted to the proximal end 176 by electrical conductor 184, such as, an electrical lead, which is housed within the bore region 174.

The probe 170 is suitable for being inserted inside a patient as part of a surgical procedure. For example, the probe 170 can be inserted through the patient's skull and inside the patient's brain as part of a neurosurgical procedure. The probe 170 is particularly well suited to neurosurgical applications by virtue of its construction. More specifically, the tube 172 and the tip 178 comprise a rigid layer formed substantially from a ceramic selected from zirconium dioxide or aluminium oxide which gives the tube and tip a rigid material property. A rigid probe is advantageous because it can be accurately guided to a target site within the brain parenchyma. In particular, the probe 170 will not be significantly deflected from the required insertion direction even when passed through virgin brain tissue or into tough matter such as brain tumours or similar tissues. The probe 170 therefore has the further advantage of not requiring any additional reinforcement during insertion.

The probe 170 is capable of measuring electrical impedance using the disc electrode 182 and therefore, it is suitable for use during surgical procedures as part of a medical imaging system. More specifically, when used as part of a medical imaging system the proximal end 176 of the probe 170 is coupled to a medical imaging system (not shown). The medical imaging system is capable of receiving an impedance measurement relating to an aspect of a patient from the probe 170 and using the impedance measurement to generate an image of the aspect. The probe 170 comprises a single electrode 182 and so the probe 170 provides a mono-polar impedance probe. To enable the mono-polar probe 170 to calculate the electrical impedance of an aspect of a patient a second conductor is required and usually comprises the patient's body, as is well known in the art.

FIG. 10 shows an alternative surgical probe 190 which provides a bi-polar impedance probe. The probe 190 differs in construction from the probe 170 of FIG. 9 in the following ways. A second disc electrode 192 is positioned within the bore region 174 and between the disc electrode 182 and the proximal end 176. Preferably, the disc electrode 192 is coaxial with the tube 172 and is positioned adjacent to the electrode 182. The second disc electrode 192 is conducted to the proximal end 176 by the electrical conductor 184. The probe 190 also differs from the probe 170 by the presence of an electrically insulating portion 194 which is positioned in-between the electrode 182 and the second electrode 192. The insulating portion 194 functions to electrically insulate the electrodes 182 and 192 from each other.

Additionally, it is within the scope of the appended claims that the outer surface of the probe 170 and the probe 190 is encoded with an absolute scale so that the probe's axial position inside a patient can be quickly and easily determined. Accordingly, each probe can be reliably guided to the correct depth within a patient.

It is within the scope of the present invention that the various embodiments described above are suitable for use with robotic equipment, preferably, robotic equipment for use in medical applications. For example, the above-described embodiments are suitable for use with tele-manipulator robotic equipment. A tele-manipulator provides a hand-like robotic mechanism which is capable of being controlled by a human operator to perform surgical operations. For example, a surgeon can remotely guide a tele-manipulator robot into a patient's central nervous system and thereby deliver to a target site within the patient an embodiment of the present invention, such as a catheter according to the present invention. The use of robotic equipment with embodiments of the present invention can be advantageous for a number of reasons. When access to a patient is restricted, for example when the patient is inside an magnetic resonance imaging (MRI) machine, robotic equipment can be built to operate within a space which is too constricted for a human to operate in effectively. Also, gearing in robotic equipment can provide improved dexterity during delivery of an embodiment according to the present invention when compared to manual delivery by a human.

1. An optical instrument for use in surgery, the instrument comprising a tube having at least one optical fibre arranged within a bore region, a wall of the tube comprising a rigid layer formed substantially from a ceramic selected from zirconium dioxide or aluminium oxide.

2. The optical instrument according to claim 1, wherein the at least one optical fibre extends between a distal end of the tube and a proximal end of the tube and is capable of transporting light between both ends.

3. The optical instrument according to claim 2, wherein the at least one optical fibre is further arranged to receive light from outside the tube at the distal end, and provide the received light at the proximal end to an image reproducing means for reproduction of an image present at the distal end.

4. The optical instrument according to claim 3, wherein at the distal end of the tube the at least one optical fibre is terminated with a substantially convex profile so that the instrument's field of view is large with respect to an outer diameter of the tube.

5. The optical instrument according to claim 3, further arranged to be coupled to a light source for delivering light to the distal end.

6. The optical instrument according to claim 2, wherein the at least one optical fibre is further arranged to emit light out of the tube from the distal end.

7. The optical instrument according to claim 6, wherein the at least one optical fibre is terminated at the distal end with a profile for causing at least one of the following effects in the emitted light:
a. diffusion which is wide with respect to an outer diameter of the tube;
b. focusing which is narrow with respect to an outer diameter of the tube; and
c. refraction at 90° to a central axis of the tube.

8. The optical instrument according to claim 6, wherein the emitted light is received from a light source coupled to the proximal end.

9. A surgical probe comprising a tube terminated at a distal end by a tip, at least a wall of the tube comprising a rigid layer formed substantially from a ceramic selected from zirconium dioxide or aluminium oxide, the probe further comprising a first electrode housed within a bore region of the tube and positioned towards the distal end.

10. The surgical probe according to claim 9, wherein the first electrode is a disc electrode which is coaxial with the tube, positioned adjacent to the tip and in electrical communication with a proximal end of the tube.

11. The surgical probe according to claim 9, further comprising a second electrode housed within the bore region, positioned the proximal side of the first electrode, wherein both electrodes are electrically insulated from each other.

12. The surgical probe according to claim 11, wherein the second electrode is a disc electrode which is coaxial with the tube, positioned adjacent to the first electrode, and in electrical communication with the proximal end.

13. A catheter for inserting into a subject comprising a tube, the wall of the tube comprising a rigid layer formed substantially from a ceramic selected from zirconium dioxide or aluminium oxide.

14. A catheter according to claim 13, wherein the catheter is a neurosurgical catheter, for insertion into the brain parenchyma of a subject.

15. A catheter according to claim 13, wherein the rigid layer comprises at least 95% by weight of the ceramic.

16. A catheter according to claim 13, wherein the ceramic layer forms or covers at least 75% of circumference of the catheter tube wall.

17. A catheter according to claim 13, wherein the outer diameter of the catheter is between 1001 im and 1.5 mm.

18. A catheter according to claim 13, wherein the tip of the catheter is rounded in shape at its end.

19. A catheter according to claim 13, wherein the outer diameter of the external wall of the catheter is reduced by one or more steps.

20. A catheter according to claim 13, wherein the outer diameter of the external wall of the catheter is reduced by tapering.

21. A catheter according to claim 13, wherein the external wall of the catheter is provided with one or more grooves at the distal end.

22. A neurosurgical kit comprising: a neurosurgical catheter according to claim 14, and a neurosurgical guide tube device, wherein the neurosurgical guide tube device comprises a guide channel through which the neurosurgical catheter can be passed.

23. A biopsy needle formed from or comprising rigid zirconium dioxide or aluminium oxide.

24. A surgical implant formed from or comprising rigid zirconium dioxide or aluminium oxide.

25. A surgical electrode comprising a rigid rod or tube made from or comprising a rigid layer of zirconium dioxide or aluminium oxide and comprising an electrically conducting material extending along the length of the tube or rod and being exposed or electrically connected to an exposed area on the surface of the rod or tube.

26. A method of manufacturing an implantable surgical device comprises the steps of extruding a rigid tube or rod of zirconium dioxide or aluminium oxide or coating a tube or rod with a rigid layer of zirconium dioxide or aluminium oxide.

27. A method of delivering a therapeutic substance to a target within the brain parenchyma of a subject comprising the steps of (i) taking a catheter, according to claim 13 and (ii) inserting the device into a subject.

28. The method of claim 27, further comprising the step (iii) of delivering a therapeutic substance to the brain parenchyma via the implanted catheter.

29. An implantable surgical device, comprising an advancement means for retracting or advancing the device or a portion of the device along an axis of insertion into a subject.

30. An advancement means for retracting or advancing at least a portion of an implantable surgical device along an axis of insertion into a subject.
